(19) United States
(12) Patent Application Publication
(10) Pub. No.: US 2012/0129210 A1
(43) Pub. Date: May 24, 2012

(54) Method for Assaying Inositol Hexaphosphate (IHP)

(76) Inventors: Vanessa Bourgeaux, Lyon (FR); Yann Godfrin, Lyon (FR)

(21) Appl. No.: 13/262,408

(22) PCT Filed: Apr. 6, 2010
(86) PCT No.: PCT/EP2010/054516

§ 371 Date: Dec. 20, 2011

(30) Foreign Application Priority Data
Apr. 3, 2009 (FR) 0952208

(51) Int. Cl. G01N 21/25; C12Q 1/02

(52) U.S. Cl. 435/29; 436/104

(57) ABSTRACT

The invention relates to a method for assaying inositol hexaphosphate (IHP) in a product that can be injected in humans or animals or in a fraction of this product, in which a metal compound is added to a sample or a fraction of this product and the complexation of said metal compound with the IHP present is subsequently detected, by virtue of which the IHP present in the product or fraction thereof is assayed. The invention makes it possible to assay the IHP in a suspension or a solution, and in particular in the various compartments of a suspension of red blood cells.

The present invention relates to a method for assaying inositol hexaphosphate or IHP in a product that can be injected in humans or animals or in a fraction of this product, in particular a suspension of red blood cells.

IHP is a powerful allosteric effector of haemoglobin. In this respect, it can be used to reduce the affinity of haemoglobin for oxygen and to increase the release of oxygen in the tissues. It can be used as a radiotherapy adjuvant in cancerology, for hypoxic tumours in particular. WO-A-2006/016247 describes the encapsulation of IHP in red blood cells serving as a biovector.

The clinical use of red blood cells encapsulating IHP requires the amount of IHP present to be known, in order to be able to determine the volume of suspension to transfuse according to the prescribed dose. In addition, since IHP is a metal-chelating agent, e.g. calcium-chelating agent, that can potentially have harmful effects if it is administered in free form, it is also essential to assay the extracellular IHP present in the final pharmaceutical product.

Various methods for assaying IHP have been described in the literature. Most of these methods have been developed in the context of the assaying of IHP present in food extracts. These methods can be classified in 3 groups according to whether they are direct, indirect or enzymatic.

Among the direct methods, mention may in particular be made of mass spectrometry, nuclear magnetic resonance, conductimetry and refractometry, which have in common the need for the sample to be analysed to be highly pure and which consequently have long implementation times.

The indirect methods are based on the ability of IHP to complex metal ions, it being possible to measure the formation of the [metal ions—IHP] complexes by colorimetry or fluorimetry. Kenan post, Ozge Tokul (Analytica Chimica Acta 558 (2006): 22-27) describes a method for indirect assaying of IHP in wheat and wheat-based products. This method is based on the reaction where the coloured complex [Fe(III)-thiocyanate] is replaced with the colourless complex [Fe(III)-phytate] in the presence of IHP. The reading is carried out by means of high performance liquid chromatography (HPLC) coupled to a UV-visible spectrophotometric detector.

It is also known from Plaami et al (Journal of the association of official analytical chemists, the association, Arlington, Va., US, 1991, 74, 32-36) a method for assaying phytic acid presents in cereals. The method disclosed is based on inductively coupled plasma atomic emission spectrometry (ICP-AES) for determination of phosphorous contained in the phytic acid.

It is also known from Park et al (Butterworth, London, 2006, 17(9), 727-732) various methods for determining the amount of phytic acid in food for children. One of the method described is a spectrophotometric method according to Latta and Eskin (J. Agric. Food. Chem., 1980, 28, 1315-1317) which requires a step of analyse by chromatography on ion exchange resins. The best method for assaying phytic acid according to Park et al is the AOAC method.

The enzymatic methods are based on the hydrolysis of IHP by enzymes called phytases; these methods are not adaptable to measuring intraerythrocytic IHP.

The methods described in the literature concerning assaying in agricultural products are not directly transposable to the case of pharmaceutical products containing IHP, in particular owing to the marked differences in composition. The presentation herein of these works cannot be regarded as giving weight to them in the assessment of patentability of the present invention.

Studies can also be found in the literature that relate to the assaying of IHP in erythrocytes using direct methods which have a certain number of drawbacks that make them unsuitable for routine assaying in the context of the manufacture of a pharmaceutical product.

S. Villa et al. (Adv Exp Med Biol. 1992; 326: 41-49) describe a simple and reliable procedure for measuring IHP in erythrocytes by HPLC. The method of analysis proposed comprises the following steps:
Preparing the samples by lysis of the erythrocytes incorporating IHP and extraction with perchloric acid,
Preparing the mobile phase and equilibrating the HPLC column,
Passing the samples through,
Measuring the IHP concentration by refractometry.

However, this method requires separation of the samples by HPLC and its implementation time is consequently very long. By way of indication, the preparation of the calibration range requires the measurement of 10 different IHP concentrations, each tested several times; the duration thereof can be estimated at 6 hours. Moreover, the detection limit of this method (0.5 mM) is altogether insufficient for quantifying the extracellular IHP present in the final products. This method does not meet the challenges of rapidity and sensitivity of the assay that are imposed by an industrial-scale production.

B. Teisseire et al. (J. Appl. Physiol. 1985 June; 58(6): 1810-7) evaluate the physiological effects of a transfusion of erythrocytes incorporating IHP in piglets. The article describes a method for measuring the intraerythrocytic IHP based on determining the concentration of intracellular phosphate before and after IHP incorporation. The difference between these two concentrations is attributed to the IHP. This very approximative method is not suitable for measuring intraerythrocytic IHP for a product for therapeutic use.

Mosca et al. (Adv Exp Med Biol. 1992; 326: 19-26) and R. Nano et al. (Adv Exp Med Biol. 1992; 326: 35-39) describe the same method of assaying intraerythrocytic IHP as that described in B. Teisseire et al., except for the fact that the phosphate concentration is in this case measured by NMR. In addition to the lack of accuracy already mentioned for the method of B. Teisseire, there is therefore also the difficulty in implementation and the prohibitive cost associated with the NMR equipment.

A first objective of the invention is to propose a rapid and reliable assay method.

A second objective of the invention is to propose such a method which can be readily automated.

A third objective of the invention is to propose such a manual or automated method which is able to fulfil the directives set by the ICH (International Conference on Harmonization).

The subject of the present invention is thus a method for assaying inositol hexaphosphate (IHP) in a product that can be injected in humans or animals or in a fraction of this product, in which a metal compound is added to a sample or a fraction of this product and the complexation of said metal compound with the IHP present is subsequently detected, by virtue of which the IHP present in the product or fraction thereof is assayed.

The expression “product that can be injected in humans or animals” is intended to mean a pharmaceutical product that may contain IHP as an active ingredient or as a contaminant. The invention is first and foremost directed towards the assaying of IHP present as an active ingredient in the product or a fraction thereof. The term “fraction” is intended to mean a part of the product that may contain IHP and the IHP content of which should be known. The invention is in particular directed towards the assaying of IHP in products formed from a suspension or from a solution of vectors responsible for delivering the IHP. They may be vectors containing or encapsulating IHP or vectors bound to IHP by any bonding, including a counterion. In one important mode, the assaying method according to the invention is applied to the assaying of total IHP in a suspension of vectors or to the assaying of IHP in one or other or both fractions represented by the extra-vector medium, in particular a supernatant or suspension liquid, and the vectors. Preferably, it involves a suspension of vectors, for example liposomes, microspheres or nanospheres, microcapsules or nanocapsules, red blood cells, etc. The invention is also directed towards the assaying of free IHP in the supernatant of a product containing IHP vectors.

According to the preferred embodiment of the invention, the complexation produces a complex, the colouration of which is different from that of the starting metal compound. The change in absorbance of the complex relative to the starting metal compound can be measured. According to one advantageous characteristic, the change in absorbance is detected by spectrophotometry.

According to one embodiment, the reagent is a coloured Fe(III) compound. It is in particular a metal compound which makes it possible to produce a phytate complex, and reference may in particular be made to F Crea et al., 2008, Coordination Chemistry Reviews 252, 1108-1120. As reagent, mention may, for example, be made of Fe(III)-thiocyanate and Fe(III)-5-sulpho-salicylic acid which will be in the form of Fe(III)-sulfosalicylate in solution. In one embodiment the reagent is Fe(III)-thiocyanate.

In one embodiment the reagent is Fe(III)-sulfosalicylate.

According to one embodiment, the assaying method is applied to the assaying of IHP in a suspension of red blood cells. The invention applies in particular to the assaying of IHP present in a suspension of red blood cells loaded with IHP and intended for the treatment of a patient. The method is applied to the assaying of total IHP, of intracellular IHP and/or of extracellular IHP. According to one embodiment, the method is applied to the assaying of IHP present in a certain volume of suspension. This assay may be used to determine the amount of suspension to be administered according to the dose of IHP prescribed for the patient, while at the same time taking into account the extracellular IHP content. According to another embodiment, the method is applied to the assaying of IHP present in the supernatant.

The method, applied to the assaying of total IHP, may in particular comprise:
lysing the red blood cells in the presence of the extracellular medium,
obtaining a fraction containing the IHP and devoid of haemoglobin and of cellular debris,
adding, to this fraction, a known amount of the metal compound resulting in the formation of a complex with the IHP present,
determining the total IHP content.

Applied to intra-erythrocytic IHP, the method may in particular comprise:
eliminating the extracellular medium, recovering the red blood cell fraction,
lysing the red blood cells,
obtaining a fraction containing the IHP and devoid of haemoglobin and of cell debris,
adding, to this fraction, a known amount of the metal compound resulting in the formation of a complex with the IHP present,
determining the intra-erythrocytic IHP content.

Applied to extracellular IHP, the method may comprise:
recovering the extracellular fraction,
adding, to this extracellular fraction, a known amount of the metal compound resulting in the formation of a complex with the IHP present,
determining the extracellular IHP content.

A similar procedure can be applied to any other type of vector containing IHP, for example liposomes or microspheres, and also to the assaying of free IHP in the supernatant of a product containing vectors bound to IHP.

If the extracellular IHP measurement is combined with the total IHP measurement of the same red blood cell suspension, the intra-erythrocytic IHP content can be deduced therefrom. The following equation can in particular be applied:

$$[ int racellularIHP ] = \frac{\begin{matrix}[ totalIHP ] \times 100 - [ extracellularIHP ] \times \\ ( 100 - haematocrit )\end{matrix}}{haematocrit}$$

According to one characteristic, the haemoglobin is removed by means of a step of precipitating the proteins in the presence of an acid. Any acid which makes it possible to precipitate proteins, including haemoglobin, can be envisaged for carrying out this extraction step. The step makes it possible to remove these proteins and the membrane debris, and to recover the small molecules such as IHP. By way of nonlimiting examples, mention may be made of: perchloric acid, trichloroacetic acid, oxalic acid, citric acid, nitric acid, hydrochloric acid, sulphuric acid and lactic acid. Hydrochloric acid is perfectly suitable. The fraction containing the IHP can be recovered by centrifugation.

According to one characteristic of the invention, after the Fe(III) coloured reagent (Fe(III)-thiocyanate or Fe(III)-sulfosalicylate) has been added, the sample to be assayed is incubated for a period of time sufficient for the Fe(III)-phytate complexes formed to be stabilized. This period of time is short, for example a period of time between approximately 5 and approximately 30 minutes is sufficient. The period of time may in particular be between approximately 10 and approximately 20 minutes, it is typically of the order of 15 minutes. The incubation is preferably carried out in the dark.

According to one preferred embodiment, the Fe(III) thiocyanate colouration reagent has the following characteristics:
from 0.1 to 1 μmol of Fe(III) per ml of reagent, in particular from 0.25 to 0.6 μmol/ml, preferably from 0.3 to 0.4 μmol/ml,
an excess of thiocyanate, in particular from 10 to 50 μmol/ml, preferably from 20 to 40 μmol/ml, even better still from 30 to 40 μmol/ml.

According to one characteristic of the invention, the Fe(III) thiocyanate reagent comprises from 0.25 to 0.6 μmol of Fe(III)/ml, and from 20 to 40 μmol of thiocyanate/ml. Preferably, the reagent comprises from 0.3 to 0.4 μmol of Fe(III)/ml, and from 30 to 40 μmol of thiocyanate/ml.

According to one preferred embodiment, the Fe(III)-sulfosalicylate colouration reagent comprises from 0.5 to 2 μmol of Fe(III)/ml and from 5 to 15 μmol of sulfosalicylate/ml.

The reagent further comprises water and is in particular made up of an acid solution, in particular with the same acid as used during the precipitation.

According to one characteristic, the reagent is a hydrochloric solution of Fe(III) and of thiocyanate.

According to one characteristic, the reagent is a hydrochloric solution of Fe(III) and of salicylate.

According to one characteristic, the concentration of acid in the reagent is between 0.01 and 0.2 molar, preferably between 0.05 and 0.15 molar.

According to one embodiment, the invention consists of a spectrophotometric method for assaying IHP based on the ability of IHP to complex Fe(III) ions. The method is based on a replacement reaction during which the Fe(III) ion present in the assaying reagent in the form of a coloured complex [Fe(III)-thiocyanate] or [Fe(III)-sulfosalicylate] will form a new colourless complex [Fe(III)-phytate] with the IHP present in the sample to be assayed. Since the absorbance peak of the [Fe(III)-thiocyanate] complex is at 460 nm, measuring the OD₄₆₀ will make it possible to determine the concentration of [Fe(III)-thiocyanate] and to deduce therefrom, via the equation summarizing the assay reaction, the concentration of IHP. Since the absorbance peak of the [Fe(III)-sulfosalicylate] complex is at 506 nm, measuring the OD₅₀₆ will make it possible to determine the concentration of [Fe(III)-sulfosalicylate] and to deduce therefrom, via the equation summarizing the assay reaction, the concentration of IHP.

According to one characteristic of the invention, the [Fe(III)-thiocyanate] or [Fe(III)-sulfosalicylate] colouration reagent is prepared extemporaneously. The assaying method can therefore integrate a step of preparing the colouration reagent before adding it to the sample to be assayed or the sample fraction.

The Fe(III)-thiocyanate or Fe(III)-sulfosalicylate reagent is advantageously prepared shortly before it is used (typically 30 minutes to an hour before) and stored in the dark until it is used.

This method can be applied to a sample representative of a batch of red blood cells encapsulating IHP, before administration to a patient, or as a production control.

A blank can be prepared with red blood cells that have been lysed-resealed (RBC-LR) in such a way as to be able to subtract the background noise linked to the red blood cell matrix.

According to one embodiment, the method comprises the preparation of an IHP calibration range; preferably, this range is prepared on the day of the assay. According to another embodiment, in place of a calibration range, a method is used in which known amounts of IHP are added to aliquots of the sample.

The assay according to the invention is perfectly suitable for the red blood cell matrix. The IHP extraction step makes it possible in particular to remove the haemoglobin, the colouration of which is capable of affecting the absorbance measurement. The addition of a blank using red blood cells that have been lysed-resealed and then treated like a sample loaded with IHP makes it possible to determine and substract the impact of the red blood cells. The choice of the assaying reagent and the use of a simple spectrophotometer makes it possible for the assay to be automated, to be fast and to be easy to implement. The assay can thus be carried out on an automatic biochemistry instrument. The method according to the invention does not require the involvement of chromatography (HPLC, ion exchange chromatography) nor NMR spectroscopy. A drastic reduction in cost and in implementation time of the assay is obtained compared with the prior techniques. About fifteen minutes are required to pass a sample through HPLC, whereas a few seconds are sufficient for acquisition of the OD on spectrophotometer. This decrease in time spent applies to each sample tested and also for each point of the calibration range that must be performed prior to the assay. The preparation of the samples also does not require a long incubation step. The total duration of the assay is consequently considerably reduced and this makes it compatible with use on an industrial scale.

Finally, the method can be carried out without any dangerous product, for example through the choice of the acid for the protein precipitation.

A subject of the invention is also a method for treating a patient with a pharmaceutical product formed from a suspension of vector (in particular liposome, microcapsule, microsphere, red blood cells, etc.) containing IHP, in particular a suspension of red blood cells encapsulating IHP, or from a solution of vector bound to IHP, for the treatment of a pathological condition that may benefit from such a treatment, comprising the following steps:
taking or preparing such a suspension or solution, in particular a suspension of red blood cells encapsulating IHP,
removing a sample representative of this suspension or of this solution,
assaying the IHP in the sample using the assaying method according to the invention,
on the basis of the concentration data, calculating the volume of the suspension or solution, in particular suspension of red blood cells, to be administered in such a way as to respect the dose prescribed for the patient,
administering this volume to said patient.

The invention will now be described in further detail using embodiments taken by way of nonlimiting examples and with reference to the figures:

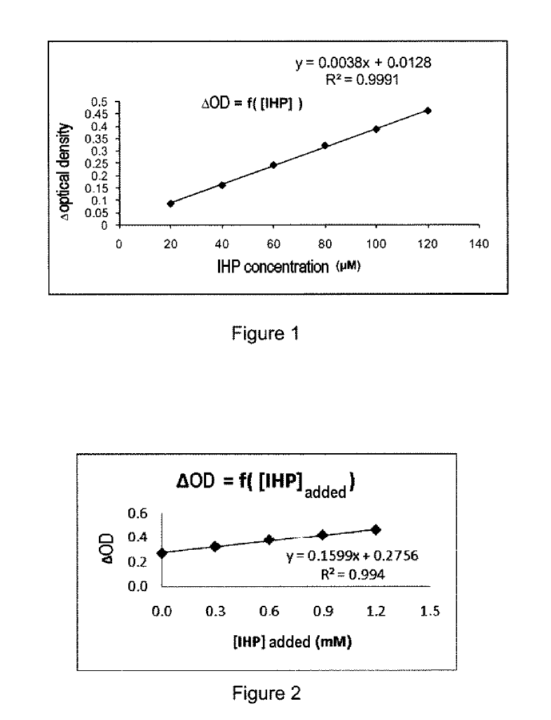

FIG. 1 is a graph representing the calibration line ΔOD as a function of the IHP concentration, obtained with the Fe(III)-thiocyanate reagent.

FIG. 2 is a graph representing the line ΔOD as a function of the concentration of IHP added, obtained according to the metered additions method, obtained with the Fe(III)-thiocyanate reagent.

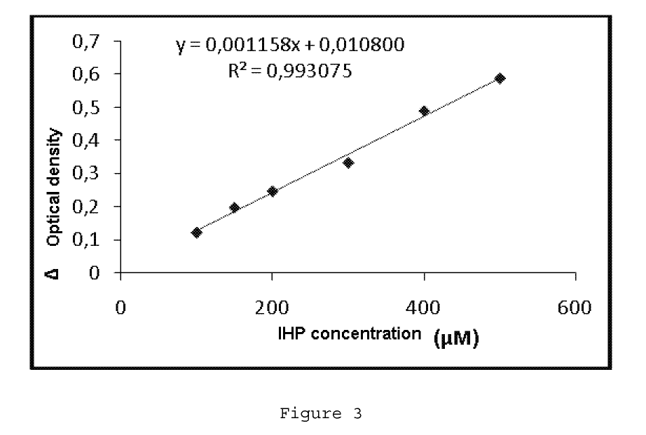

FIG. 3 is a graph representing the calibration line ΔOD as a function of the IHP concentration, obtained with the Fe(III)-sulfosalicylate.

### EXAMPLE 1

### Reagents and Chemical Solutions

Preparation of a solution of iron(III) chloride: 20 mg of iron(III) chloride are dissolved in 40 ml of distilled water (0.5 mg/ml).

Preparation of a solution of ammonium thiocyanate: 200 mg of ammonium thiocyanate are dissolved in 40 ml of distilled water (5 mg/ml).

The colouration reagent is prepared at least 40 min before use and is composed of 1 ml of iron(III) chloride at 0.5 mg/ml, 5 ml of ammonium thiocyanate at 5 mg/ml, 0.9 ml of 1N hydrochloric acid and 2 ml of distilled water.

Preparation of trichloroacetic acid TCA solutions (weight/volume)

18.75% solution: 3 ml of 6.1N TCA(100%)+13 ml of distilled water

9.375% solution: 10 ml of 18.75% TCA solution+10 ml of distilled water

7.5% solution: 15 ml of 6.1N TCA(100%)+185 ml of distilled water

24% solution: 6 ml of 6.1N TCA(100%)+19 ml of distilled water

12% solution: 10 ml of 24% TCA solution+10 ml of distilled water

6% solution: 15 ml of 6.1N TCA(100%)+235 ml of distilled water

Preparation of a stock solution of IHP: for 1 ml of solution at 45 mM, the amount X of the dodecasodium salt of IHP to be weighed out is calculated in the following way: X (g)=415.719/(p×(100−H)), p being the purity of the salt (%) and H the water content (%). The final volume is adjusted to 1 ml with distilled water.

Method of encapsulating IHP in human red blood cells: the IHP (660 g/mol) was encapsulated in human red blood cells (RBCs) by the hypotonic column dialysis method. The RBCs from a bag are washed 3 times in 0.9% NaCl. The haematocrit is brought to 60% in the presence of IHP added at a final concentration of 17 or 20 mM, before starting the dialysis. The RBCs are dialysed at a flow rate of 1.5 ml/min against a low-osmolarity lysis buffer (counterflow at 15 ml/min). The lysed RBCs exiting the column are resealed by means of the addition of a hyperosmolar solution and incubation for 30 minutes at 37° C. After several washes in 0.9% NaCl, 0.2% glucose, the RBCs are brought to a haematocrit of 50% with a storage solution (AS-3 buffer).

### EXAMPLE 2

### Assaying of IHP in Total RBC-IHP Samples and in the Extracellular Medium

### 1) Nature of the Samples to be Assayed

In order to determine the intracellular IHP, the total IHP in the sample (RBC-IHP) and also the IHP content in the supernatant (extracellular medium) are assayed. The supernatant is obtained by centrifugation of RBC-IHP at 1000 g, 4° C., for 10 min. The sample to be assayed may also be an aqueous solution of IHP which will then not have to undergo any extraction.

Assaying Method with the Calibration Range (External Calibration)

### Sample Preparation

The RBC-IHPs (50 μl) and the supernatants (50 μl) are frozen at −20° C. for 30 min. After reheating to ambient temperature, the samples are diluted with distilled water in order to lyse the cells, and the IHP is extracted under acidic conditions with trichloroacetic acid. Depending on the nature of the sample, the dilution applied varies. Various concentrated solution of trichloroacetic acid (TCA) can be used for the preparation of the sample.

Each reagent is added as indicated in the following table:

| | Volume of | Volume of |
|---|---|---|
| Sample | distilled water | trichloroacetic acid |
| Supernatant | 125 μl | 175 μl of 9.375% |
| | | trichloroacetic acid |
| RBC-IHP | 950 μl | 333 μl of 18.75% |
| | | trichloroacetic acid |

The samples are then centrifuged at 15 000 g, 4° C., for 10 min and the extraction supernatant is collected for bringing into contact with the colouration reagent. A further dilution is carried out for the RBC-IHPs (75 μl of 7.5% trichloroacetic acid are mixed with 75 μl of the extraction supernatant) before mixing with the colouration reagent.

Preparation of the IHP Calibration Range

Preparation of a 1 mM Solution

20 μl of the solution of IHP at 45 mM (see Example 1) are mixed with 880 μl of 7.5% trichloroacetic acid.

Preparation of the Standard Solutions:

| | Final concentration | | | | | |
|---|---|---|---|---|---|---|
| | of IHP (μM) | | | | | |
| | 20 | 40 | 60 | 80 | 100 | 120 |
| Volume of 1 mM IHP (μl) | 20 | 40 | 60 | 80 | 100 | 120 |
| Volume of 7.5% | 980 | 960 | 940 | 920 | 900 | 880 |
| trichloroacetic acid (μl) | | | | | | |

The blank is made of 7.5% trichloroacetic acid.

Photometric Detection

For the photometric detection, 300 μl of the colouration reagent (prepared as in Example 1) are added to 150 μl of each extraction supernatant and standard solution and to the blank. After agitation and incubation for 15 min in the dark, the solutions are placed in 10 mm cuvettes and the absorption is measured at 460 nm. For each concentration (20-120 μM), the delta absorbance ΔOD=OD_{blank}−OD_{standard} is determined. The ΔOD/[IHP] calibration curve is plotted and the linear regression parameters are calculated and used to determine the amount of IHP present in the extraction supernatants by virtue of the ΔOD=OD_{blank}−ODₛₐₘₚₗₑ measurement. For the RBC-IHPs, the noise due to the interference from the red blood cell matrix is evaluated through the assaying of red blood cells having undergone the same dialysis process and being adjusted to a haematocrit of 50% under the same conditions (RBC-LR). For the RBC-IHP supernatants, no interference with the assaying was observed.

### 2) Assaying Method by Metered Additions

This method makes it possible to do away with the red blood cell matrix effect and therefore to avoid the production of RBC-LR for assaying the IHP contained in the RBC-IHPs. The sample to be assayed containing X mM of IHP is aliquoted in 50 μl volumes and various additions of IHP (950 μl) of known concentration (C1, C2, C3, C4, C5, etc.) are made during the red blood cell lysis step. The addition of 18.75% TCA (333 μl) to each of the tubes, followed by centrifugation at 15 000 g for 10 min, makes it possible to recover the IHP in the supernatants. For the photometric determination, 300 μl of the colouration reagent (prepared as in Example 1) are added to 150 μl of each extraction supernatant. A reference tube is prepared by adding 300 μl of the colouration reagent to 150 μl of 6% TCA. After agitation and incubation for 15 min in the dark, the solutions are placed in 10 mm cuvettes and the absorption is measured at 460 nm. For each point, the delta absorbance ΔOD=OD_{reference}−ODₛₐₘₚₗₑ is determined. The calibration curve of ΔOD/[IHP] added is plotted. If the curve is not a linear straight line, the highest concentration points are diluted in 7.5% trichloroacetic acid and reassayed. The point of intersection between the straight line and the x-axis (in absolute value) directly gives the value X of IHP contained in the initial sample.

### EXAMPLE 3

### Calculation of the Amount of IHP Encapsulated in the Red Blood Cells

The concentration of IHP in the RBC-IHPs and in the extracellular medium is determined as in Example 2. To calculate the intracellular IHP, the following equation is applied:

$$\frac{\begin{matrix}[ int racellularIHP ] = \\ ( {[ IHP ]}_{total} \times 100 - {[ IHP ]}_{extracellular} \times ( 100 - haematocrit ) )\end{matrix}}{haematocrit}$$

### EXAMPLE 4

### Optimization and Characterization of the Effectiveness of the Method

### 1) Stability of the Colouration Reagent

The colouration reagent was prepared as indicated in Example 1. Its stability was studied by photometry at 460 nm for 300 minutes. The results show that it is preferable to prepare it at least 40 min before use, from which time it is stable for at least 4 h, which makes it possible to carry out all the analyses.

### 2) Linearity of the Range

The linearity of the IHP range was studied over a range of from 20 to 120 μM IHP (concentration before bringing into contact with the colouration reagent). 150 μl of each range point, prepared as in Example 2, were mixed with 300 μl of the colouration reagent and the range was plotted as indicated in Example 2. The Fisher test showed that the calibration range is valid.

| | | | |
|---|---|---|---|
| | Value | Critical value | |
| Designation | observed | with α = 1% | Conclusion |

Calibration

| | | | |
|---|---|---|---|
| Number of levels | 6 | | |
| Total number of | 30 | | |
| measurements | | | |
| Sensitivity | 0.0044 | | |
| (slope) | | | |
| Blank (y-axis at | 0.0106 | | |
| the origin) | | | |
| Determination | 0.9995 | Recommendation, | Acceptable |
| coefficient | | ICH: R2 ≧ 0.995 | |

Linearity

| | | | |
|---|---|---|---|
| F of the | 15494.29 | 7.82 | Model |
| regression model | | | acceptable |
| F of the | 1.01 | 4.22 | Calibration |
| calibration model | | | range |
| | | | validated |

### 3) Optimization of the Sample Extraction Conditions

The conventional extraction using 2.5 volumes of distilled water and 3.5 volumes of 9.375% TCA was optimized in order to obtain optimized overlap percentages. A larger volume of non-osmolar aqueous solution (19 volumes) containing various IHP contents was used to simulate the extraction of suspensions of RCB+IHP at various IHP concentrations. The use of concentrated 18.75% TCA used at a final concentration of 7.5% makes it possible to not dilute the samples too much and avoids loss of assay sensitivity. The samples were assayed according to the protocol described in Example 2. The results comparing the two extraction methods are given hereinafter.

| Initial | Conventional | | | |
|---|---|---|---|---|
| concentration | extraction | | Optimized extraction | |
| of IHP in the | Concentration | % | Concentration | % |
| sample (μM) | found (μM) | overlap | found (μM) | overlap |
| 0 | | | | |
| 533 | 461 | 87% | 568 | 107% |
| 1067 | 963 | 90% | 1171 | 110% |
| 1334 | 1136 | 85% | 1446 | 108% |
| 1600 | 1387 | 87% | 1736 | 109% |
| 2134 | 1780 | 83% | 2344 | 110% |
| 2400 | 2025 | 84% | 2619 | 109% |
| 2667 | 2269 | 85% | 2459 | 92% |
| 3200 | 2555 | 80% | 3430 | 107% |

### 4) Robustness of the Method

A sample was assayed at various temperatures (6° C., 22° C. and 35° C.). The method was found to be robust with respect to changes in temperature.

### 5) Quantification and Detection Limits

The quantification and detection limits were determined according to the recommendations of the ICH. For this, eight blanks (7.5% TCA) were prepared and the range was studied in its linearity range (20 to 120 μM). After mixing with the colouration reagent, the blanks and the range points were analysed on a spectrophotometer as described in Example 2. The standard deviation of the ODs obtained was calculated so as to be able to determine LD and LQ according to the following ICH formulae:

LD=3.3σ/P LQ=10σ/P

with: σ=standard deviation based on the blank
P=slope of the calibration line

The range obtained has a slope of 0.0045 uOD/μM (uOD=unit of OD). The standard deviation of the results obtained by this method is 0.00477 uOD; after calculation, the LD and LQ values on the range are respectively 3.5 μM and 10.6 μM.

The dilution effect of the method is taken into account and, after correction, the following values are obtained:
supernatant LD=25 μM; LQ=74 μM
total LD=94 μM; LQ=283 μM.

### EXAMPLE 5

### 1) Example of a Result Using the Method with a Calibration Range and Elimination of the Noise Linked to the Red Blood Cell Matrix by Assaying the RBC-LR Matrix

Plot of the IHP calibration straight line (FIG. 1) using the values in the table below:

| Concentration | | | | |
|---|---|---|---|---|
| of IHP (μm) | OD 460 nm | ΔOD | Mean ΔOD | CV |
| 0 | 0.909 | Mean | | |
| | 0.915 | 0.912 | | |
| 20 | 0.825 | 0.087 | 0.086 | 1.64% |
| | 0.827 | 0.085 | | |
| 40 | 0.754 | 0.158 | 0.16 | 1.77% |
| | 0.75 | 0.162 | | |
| 60 | 0.669 | 0.243 | 0.242 | 0.58% |
| | 0.671 | 0.241 | | |
| 80 | 0.589 | 0.323 | 0.3205 | 1.10% |
| | 0.594 | 0.318 | | |
| 100 | 0.528 | 0.384 | 0.386 | 0.73% |
| | 0.524 | 0.388 | | |
| 120 | 0.451 | 0.461 | 0.46 | 0.00% |
| | 0.451 | 0.461 | | |

Calculation of the IHP in the samples:

| | | | | IHP | | |
|---|---|---|---|---|---|---|
| | | | | concentration | Mean IHP | |
| | Dilution | | | in | concentration | |
| | factor | | Concentration | the | estimated | |
| | due to | | on the | initial | in the | IHP |
| | the | | curve | sample | sample | concentration |
| Sample name | extraction | ΔOD | (μm) | (mM) | (mM) | (mM) |
| RBC-LR | 7 | 0.170 | 41.4 | 0.290 | 0.290 | |
| matrix | | | | | | |
| RBC-IHP | 26.67 | 0.448 | 114.5 | 3.054 | 3.037 | 2.81 |
| | 26.67 | 0.443 | 113.2 | 3.019 | | |
| Supernatant | 7 | 0.118 | 27.7 | 0.194 | 0.230 | 0.23 |
| | 7 | 0.085 | 19.0 | 0.266 | | |

### 2) Example of a Result Using the Metered Additions Method

The sample to be assayed was aliquoted in 50 μl volumes and treated as described in detail in Example 2 (metered additions method). The table of the additions made is the following:

| Addition | C1 | C2 | C3 | C4 | C5 |
|---|---|---|---|---|---|
| Concentration of the | 0 | 0.0157 | 0.0314 | 0.0474 | 0.0632 |
| solution added (mM) | | | | | |
| Concentration | 0 | 0.3 | 0.6 | 0.9 | 1.2 |
| corresponding to the | | | | | |
| addition to the | | | | | |
| initial sample (mM) | | | | | |

The points obtained (table below) made it possible to plot the straight line of FIG. 2.

| [IHP] added (mM) | ΔOD | CV |
|---|---|---|
| 0 | 0.271 | 5% |
| 0.3 | 0.324 | 1% |
| 0.6 | 0.381 | 2% |
| 0.9 | 0.420 | 1% |
| 1.2 | 0.463 | 3% |

The concentration is determined at the point y=0, which gives [IHP] in absolute value in the initial sample=1.72 mM. It is calculated using the equation: y=1.599x+0.2756.

### 3) Comparison of Metered Additions and Calibration Range Method

| | Assay with calibration | Metered additions | |
|---|---|---|---|
| Test | range (mM) | assay (mM) | % CV |
| 1 | 1.64 | 1.72 | 3% |
| 2 | 1.86 | 1.92 | 2% |
| 3 | 1.84 | 1.82 | 1% |

### EXAMPLE 6

### Automation of the Assay

Example with the Assay by Calibration Range

The assaying of RBC encapsulating IHP was partially automated on a biochemistry instrument (MaxMat). The colouration reagent (reagent), the 7.5% TCA (diluent) and the 1 mM IHP stock solution (standard) were prepared as in Example 1, and then placed on the platform of the automated device. The samples to be assayed were prepared manually according to the process described in Example 2. Each supernatant from extraction of the samples to be assayed was transferred into a 1.5 ml tube and placed on the platform.

The assaying method was correctly parameterized:
positive mode
negative linear regression mode
30 cycles:
   cycle 1: sampling of the sample prepared: 75 μl and OD reading at 460 nm
   cycle 2: addition of the colouration reagent: 150 μl
   cycle 30: OD reading at 460 nm.

When the assay is initiated, the automated device prepares the dilutions of the calibration range (1/8.3, 1/10, 1/12, 1/15, 1/25, 1/50) in 7.5% TCA, and then the various mixtures with the colouration reagent. When the 30 cycles are complete, the automated device indicates an OD value at 460 nm for the range points and the various samples. The results are then transferred to an excel calculation sheet identical to that of Example 3. The assaying of one or more samples is carried out in less than 60 min.

The results obtained for several samples by manual and automated assaying are comparable (% CV<10%) for the RBC-IHPs at a 50% haematocrit and the supernatants (see table below).

| | | Automated | Manual assay | | |
|---|---|---|---|---|---|
| | | assay mean | mean | | |
| | | concentration | concentration | | |
| | Nature of | of IHP in the | of IHP in the | | |
| Sample | the sample | sample (mM) | sample (mM) | Mean | CV |
| 1 | RBC-IHP | 2.933 | 2.781 | 2.857 | 4% |
| 2 | Supernatant | 0.218 | 0.203 | 0.211 | 5% |
| 3 | Supernatant | 0.564 | 0.543 | 0.554 | 3% |
| 4 | Supernatant | 0.387 | 0.431 | 0.409 | 8% |

### EXAMPLE 7

### Assay with the Fe(III)-Sulfosalicylate as Ligand and Comparison with the Fe(III)-Thiocyanate

a) Description of the Method

Preparation of a solution of iron(III) chloride: 49 mg of iron (III) chloride were dissolved in 21.2 ml of distilled water (2.31 mg/ml).

Preparation of a solution a 5-sulfosalicylic acid: 98.6 mg of sulfosalicylate were dissolved in 19.7 ml of distilled water (5 mg/ml).

Preparation of a solution of hydrochloric acid: 500 μl of hydrochloric acid 1N were dissolved in 9.5 ml of distilled water (0.05N).

The colouration reagent was composed of 1 ml of the 2.31 mg/ml iron(III) chloride solution, 5 ml of the 5 mg/ml sulfosalicylic acid solution, 0.9 ml of the 0.05N of hydrochloric acid solution and 2 ml of distilled water.

The Fe(III)-Thiocyanate was prepared according to the method mentioned above.

Sample Preparation:

The RBS-IHP (50 μl) and the supernatants (50 μl) were frozen at −20° C. for 30 min. After reheating to ambient temperature, the samples were diluted with distilled water in order to lyse the cells, and the IHP was extracted under acidic conditions with trichloroacetic acid. Depending on the sample, the dilution applied varied. Each reagent was added as indicated in the following table:

| | Volume of | Volume of |
|---|---|---|
| Sample | distilled water | trichloroacetic acid |
| Supernatant IHP | 125 μl | 175 μl of 12% |
| | | trichloroacetic acid |
| RBC-LR | 125 μl | 175 μl of 12% |
| | | trichloroacetic acid |
| RBC-IHP | 325 μl | 125 μl of 24% |
| | | trichloroacetic acid |

The sample were then centrifuged at 15 000 g, 4° C., for 10 min and the extraction supernatant was collected for bringing into contact with the colouration agent.

Photometric Detection

For the photometric detection, 300 μl of the colouration agent were added to 150 μl of each extraction supernatant and standard solution and to the blank. After agitation and incubation for 15 minutes in the dark, the solutions were placed in 10 mm cuvettes and the adsorption was measured at 506 nm. For each concentration (100-500 μM), the delta absorbance ΔOD=OD_{blank}−OD_{standard} was determined. The ΔOD/[IHP] calibration curve was plotted and the linear regression parameters were calculated and used to determine the amount of IHP present in the extraction supernatants by virtue of the ΔOD=OD_{blank}−ODₛₐₘₚₗₑ measurement. For the RBC-IHPs, the noise due to the interference from the red blood cell matrix was evaluated through the assaying of red blood cells having undergone the same dialysis process without colouration reagent and being adjusted to a haematocrit of 50% under the same conditions (RBC-LR).

b) Preparation of the IHP Calibration Range

Preparation of a 1 mM Solution

20 μl of the solution of IHP at 45 mM (see example 1) were mixed with 880 μl of 6% trichloroacetic acid.

Preparation of the Standard Solutions:

| | Final concentration | | | | | |
|---|---|---|---|---|---|---|
| | of IHP (μM) | | | | | |
| | 100 | 150 | 200 | 300 | 400 | 500 |
| Volume of 1 mM IHP (μl) | 100 | 150 | 200 | 300 | 400 | 500 |
| Volume of 6% trichloroacetic | 900 | 850 | 800 | 700 | 600 | 500 |
| acid (μl) | | | | | | |

The blanck was made of 6% trichloroacetic acid.

FIG. 3 represents the calibration line ΔOD as a function of the IHP concentration, obtained with Fe (III)-sulfosalicylate.

The method is linear since the R² value equals 0.99075.

c) Example of Assaying with the Calibration Range

Assaying of Total Sample

The total sample relates to the assaying of total IPH contained in the final products (RBC at hematocrit 50%).

The method used was explained above.

Complex used

| Assaying | FeIII/ | FeIII/ | % CV between |
|---|---|---|---|
| sample | thiocyanate | sulfocalycilic | the 2 methods |
| 1 | 2.46 mM | 2.59 mM | 4% |
| 2 | 2.69 mM | 2.61 mM | 2% |
| 3 | 2.55 mM | 2.68 mM | 4% |
| 4 | 3.38 mM | 3.64 mM | 5% |

The two methods gave similar results.

Assaying of Sample Containing Great and Known Amount of IHP

| | | | | Difference |
|---|---|---|---|---|
| | Measured IHP | | Difference | compared to |
| | concentration (mM) | | compared to the | the theoretical |
| | FeIII/ | FeIII/ | theoretical value | value with |
| | thio- | sulfo- | with FeIII/ | FeIII/sulfo- |
| | cyanate | salicylate | thiocyanate | salicylate |
| GR + IHP | 10.24 | 8.10 | 2.4% | 22.8% |
| at 10.5 mM | | | | |
| GR + IHP | 16.24 | 17.80 | 3.5% | 4.7% |
| at 17 mM | | | | |

Assaying of IHP in the Supernatants

Measured IHP
concentration (mM)

| | FeIII/ | FeIII/ | |
|---|---|---|---|
| | thiocyanate | sulfosalicylate | CV (%) |
| Matrice s-GRLR | / | 0.308 | |
| Supernatant 1 | 0.272 | 0.298* | 6% |
| Supernatant 2 | 0.36 | 0.414* | 10% |
| Supernatant 3 | 0.502 | 0.587* | 11% |

*values obtained by substracting the background noise.

The method using the Fe(III)/sulfosalicylate showed a backround noise elevated for assaying the supernatant (until 50% of the value evaluated by colorimetric method).

Estimating of the IPH Amount Contained in RBC

Complex nature

| | FeIII/ | FeIII/ | |
|---|---|---|---|
| | thiocyanate | sulfocalycilate | CV (%) |
| GR-IHP 1 | 19.04 μmol/g Hb | 19.99 μmol/g Hb | 3% |
| GR-IHP 2 | 25.03 μmol/g Hb | 26.93 μmol/g Hb | 5% |

The values obtained by with the two complex are similar.

Quantification and Detection Limits for the Fe(III)-Sulfosalicylate Method

The range obtained has a slope of 0.001158 uOD/μM (uOD=unit of OD). The standard deviation of the results obtained by this method was 0.0001737 uOD; after calculation the LD and LQ values on the range were respectively 22 μM and 67 μM.

The dilution effect of the method is taken into account and, after correction, the following values were obtained:
supernatant LD=154 μM; LQ=469 μM
total LD=220 μM; LQ=670 μM

1. Method for assaying inositol hexaphosphate (IHP) in a product that can be injected in humans or animals or in a fraction of this product, in which a metal compound is added to a sample or a fraction of this product and the complexation of said metal compound with the IHP present is subsequently detected, by virtue of which the IHP present in the product or fraction thereof is assayed.

2. Method according to claim 1, in which the complexation produces a complex, the colouration of which is different from that of the metal compound.

3. Method according to claim 2, in which the change in absorbance of the complex relative to the starting metal compound is measured.

4. Method according to claim 3, in which this change in absorbance is detected by spectrophotometry.

5. Method according to claim 1, in which the reagent is an Fe(III) compound.

6. Method according to claim 5, in which the Fe(III) compound is Fe(III)-thiocyanate.

7. Method according to claim 5, in which the Fe(III) compound is Fe(III)-sulfosalicylate.

8. Method according to claim 1, in which the sample is a suspension of red blood cells.

9. Method according to claim 8, applied to total IHP, the method comprising:
lysing the red blood cells,
obtaining a fraction containing the IHP and devoid of haemoglobin and of cell debris,
adding, to this fraction, a known amount of the metal compound resulting in the formation of a complex with IHP present,
determining the total IHP content.

10. Method according to claim 8, applied to intra-erythrocytic IHP, the method comprising:
eliminating the extracellular medium, recovering the red blood cell fraction,
lysing the red blood cells,
obtaining a fraction containing the IHP and devoid of haemoglobin and of cell debris,
adding, to this fraction, a known amount of the metal compound resulting in the formation of a complex with the IHP present,
determining the intra-erythrocytic IHP content.

11. Method according to claim 9, in which the haemoglobin is removed by means of a step of precipitating the proteins in the presence of an acid.

12. Method according to claim 8, applied to extracellular IHP, the method comprising:
recovering the extracellular fraction,
adding, to this extracellular fraction, a known amount of the metal compound resulting in the formation of a complex with IHP present,
determining the extracellular IHP content.

13. Method according to claim 12, and further including the step of
measuring the total IHP, including the steps of
lysing the red blood cells,
obtaining a fraction containing the IHP and devoid of haemoglobin and of cell debris,
adding, to this fraction, a known amount of the metal compound resulting in the formation of a complex with IHP present, and
determining the total IHP content; and
wherein the intra-erythrocytic IHP content being obtained by use of the following equation:
$$[ int racellularIHP ] = \frac{\begin{matrix}[ totalIHP ] \times 100 - [ extracellularIHP ] \times \\ ( 100 - haematocrit )\end{matrix}}{haematocrit}$$
